(19) United States
(12) Patent Application Publication
(10) Pub. No.: US 2025/0325045 A1
(43) Pub. Date: Oct. 23, 2025

(54) Catheter-Accessible Trousers with Integrated Fastener

(71) Applicant: Sally Nason, Scarborough, ME (US)

(72) Inventor: Sally Nason, Scarborough, ME (US)

(21) Appl. No.: 18/958,133

(22) Filed: Nov. 25, 2024

Related U.S. Application Data
(60) Provisional application No. 63/637,397, filed on Apr. 23, 2024.

(51) Int. Cl. A41D 1/06; A41D 27/20; A61F 5/44

(52) U.S. Cl. CPC A41D 1/06; A41D 27/20; A61F 5/4408

(57) ABSTRACT

The present invention is a catheter-friendly pair of pants designed to facilitate the use of catheters for individuals. The pants include a first leg member and a second leg member extending from a waist member, with an inner seam zipper integrated into at least one leg member. The zipper, ranging from 6 to 12 inches, enables a catheter tube to pass therethrough without causing discomfort and is designed to remain out of sight, preserving the appearance of regular pants. In some embodiments, the pants include an interior pocket with a transparent inspection window for accommodating and monitoring a catheter bag. The fastener options include zippers, hook and loop, snap-buttons, pressure adhesives, and magnetic fasteners, ensuring flexibility and user convenience.

### CROSS-REFERENCE TO RELATED APPLICATION

The present application claims priority to, and the benefit of, U.S. Provisional Application No. 63/637,397, which was filed on Apr. 23, 2024 and is incorporated herein by reference in its entirety.

### FIELD OF THE INVENTION

The present invention generally relates to the field of garments. More specifically, the present invention relates to a novel catheter-friendly pair of pants designed to facilitate the use of catheters for individuals with chronic bladder conditions, spinal cord injuries, or post-surgical needs. The pants enable easy access of a catheter tube and includes an inner seam zipper integrated into inner seam of a leg member. The zipper enables a catheter tube to pass through without causing discomfort and is designed to remain out of sight, preserving the appearance of regular pants. Accordingly, the present disclosure makes specific reference thereto. Nonetheless, it is to be appreciated that aspects of the present invention are also equally applicable to other like applications, devices, and methods of manufacture.

### BACKGROUND

By way of background, many individuals require catheters to rest the bladder after surgery, particularly following procedures involving the bladder, bowel, or urinary tract. A catheter is a tube inserted into the bladder, enabling urine to drain freely into an external bag. However, managing the catheter bag and tube can be challenging, especially when the bag needs to be emptied.

When wearing long pants, individuals often have to thread the catheter and tubing through the pant leg and out of the bottom end, where the catheter bag is then attached to a wheelchair or another support. This conventional setup can interfere with proper urine flow due to potential bends in the tubing, causing discomfort and possible blockages. Additionally, accessing the catheter often requires the wearer to remove their pants completely, which is inconvenient and can lead to a loss of privacy.

Current solutions include smaller leg bags that can be concealed within pant legs, but smaller bags are required to be drained more frequently than larger bags, adding to the inconvenience and reducing the overall practicality of the system. Individuals desire a more user-friendly solution that provides easy access to the catheter and maintains proper urine flow.

Therefore, there exists a long felt need in the art for improved trousers that provide easy access to the catheter. There is also a long felt need in the art for catheter-friendly trousers that enable a catheter tube to pass through an opening for easy accessibility. Additionally, there is a long felt need in the art for special trousers that help in maintaining proper urine flow in a catheter tube. Moreover, there is a long felt need in the art for a uniquely designed pair of pants that feature an inner seam zipper that enables a catheter tube to easily exit the pants. Further, there is a long felt need in the art for a trousers garment that can hold a catheter bag and provides easy access to the bag. Furthermore, there is a long felt need in the art for trousers that includes an inner seam zipper, which enables the catheter tube to exit the pants conveniently. Finally, there is a long felt need in the art for a uniquely designed garment that enables for easy access to a catheter tube and bag while maintaining comfort, privacy, and convenience.

The subject matter disclosed and claimed herein, in one embodiment thereof, comprises a catheter-friendly pair of pants. The pair of pants include a first leg member and a second leg member, each extending vertically downwards from a waist member. An inner seam runs along the length of at least one of the leg members, a fastener, preferably a zipper is integrated into the inner seam of the first leg member. The fastener has a length of approximately from 6 to 12 inches and extends from a top end near a thigh portion to a bottom end near a knee covering portion. An interior pocket is disposed on the inner surface of the first leg member for accommodating a catheter bag, wherein the interior pocket includes a transparent member for functioning as an inspection window, enabling users to check the bag's fill level without needing to remove the bag from the pants.

In this manner, the catheter-friendly pair of pants of the present invention accomplishes all of the forgoing objectives and provides users with a novel garment that addresses the practical needs of catheter users. A strategically placed inner seam zipper enables for easy access to the catheter tube and bag while maintaining comfort, privacy, and convenience. The larger catheter bag reduces the frequency of maintenance, enhancing the user's independence and quality of life. The pair of pants is a user-friendly solution that provides easy access to the catheter, maintains proper urine flow, and enables for discretion and comfort in daily activities.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a catheter-friendly pair of pants. The pair of pants include a first leg member and a second leg member, each leg member extends vertically downwards from a waist member. A zipper is integrated into the inner seam of at least one of the leg members, the zipper having a length between 6 inches and 12 inches, extending from a top end near a thigh portion to a bottom end near a knee covering portion. The zipper is configured to be unfastened for providing space for a catheter tube to pass through without causing discomfort to a wearer.

In yet another embodiment, catheter-friendly pair of pants are disclosed. The pair of pants include a first leg member and a second leg member, each extending vertically downwards from a waist member, an inner seam running along the length of at least one of the leg members, a fastener is integrated into the inner seam of the first leg member, the fastener has a length of approximately from 6 to 12 inches and extends from a top end near a thigh portion to a bottom end near a knee covering portion. An interior pocket is disposed on the inner surface of the first leg member for accommodating a catheter bag, wherein the interior pocket includes a transparent member for functioning as an inspection window, enabling users to check the bag's fill level without needing to remove the bag from the pants.

In another embodiment, a catheter-friendly pair of pants are disclosed. The pair of pants include a first leg member and a second leg member, each extending vertically downwards from a waist member, an inner seam running along the length of at least one of the leg members, a fastener integrated into the inner seam of the first leg member, selected from the group consisting of a zipper, a hook and loop fastener, a snap-button fastener, a pressure adhesive fastener, and a magnetic fastener, providing an opening for guiding a catheter tube to exit the leg member, wherein the pants are made from antimicrobial and moisture-wicking fabrics selected from the group consisting of cotton, polyester, denim, wool, corduroy, leather, and blends thereof, wherein the fastener is configured to be maintained out of sight, thereby maintaining the appearance of regular pants and keeping the medical condition of the wearer private.

In another aspect, the interior pocket is made of a material selected from the group consisting of natural materials such as cotton and hemp, and synthetic materials such as polymers and sponges, providing durability and comfort for the catheter bag placement.

In yet another aspect, the hook and loop fastener is configured to enable partial fastening, securing the catheter tube in place while leaving space for the tube to move freely, thus preventing the tube from being pinched and ensuring the wearer's comfort.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the catheter-friendly pair of pants of the present invention in accordance with the disclosed structure;

FIG. 2 illustrates a perspective view showing use of the catheter-friendly pair of pants of the present invention in accordance with the disclosed structure;

FIG. 3 illustrates a perspective view showing another embodiment of the catheter-friendly pair of pants of the present invention in accordance with the disclosed structure;

FIG. 4 illustrates an enlarged view of the inner seam zipper used in the pair of pants in accordance with the disclosed structure; and

FIG. 5 illustrates a perspective view showing another embodiment of fastener used in the catheter-friendly pair of pants of the present invention in accordance with the disclosed structure.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for improved trousers that provide easy access to the catheter. There is also a long felt need in the art for catheter-friendly trousers that enable a catheter tube to pass through an opening for easy accessibility. Additionally, there is a long felt need in the art for special trousers that help in maintaining proper urine flow in a catheter tube. Moreover, there is a long felt need in the art for a uniquely designed pair of pants that feature an inner seam zipper that enables a catheter tube to easily exit the pants. Further, there is a long felt need in the art for a trousers garment that can hold a catheter bag and provides easy access to the bag. Furthermore, there is a long felt need in the art for trousers that includes an inner seam zipper, which enables the catheter tube to exit the pants conveniently. Finally, there is a long felt need in the art for a uniquely designed garment that enables for easy access to a catheter tube and bag while maintaining comfort, privacy, and convenience.

The present invention, in one exemplary embodiment, is a catheter-friendly pair of pants. The pair of pants include a first leg member and a second leg member, each extending vertically downwards from a waist member, an inner seam running along the length of at least one of the leg members, a fastener is integrated into the inner seam of the first leg member, the fastener has a length of approximately from 6 to 12 inches and extends from a top end near a thigh portion to a bottom end near a knee covering portion. An interior pocket is disposed on the inner surface of the first leg member for accommodating a catheter bag, wherein the interior pocket includes a transparent member for functioning as an inspection window, enabling users to check the bag's fill level without needing to remove the bag from the pants.

Reference will now be made in detail to the present preferred embodiments of the invention, examples of which are illustrated in the accompanying drawings. Wherever possible, the same reference numerals are used in the drawings and the description to refer to the same or like parts.

The term “fastener” in the present disclosure describes a device for attaching or connecting two items together in a manner that also enables for the detachment of the two items. The fastener can be readily reusable in that its normal securing and releasing operation is not destructive or damaging to the fastener itself or to the items of which it connects.

The term “open” in the present disclosure refers to the ability, capability or state of something being put in a position to free, clear, unblock, make accessible, make available, enable access to or passage across, an entrance, gap, passage, aperture, and/or opening; it also means to separate, detach, disconnect two items.

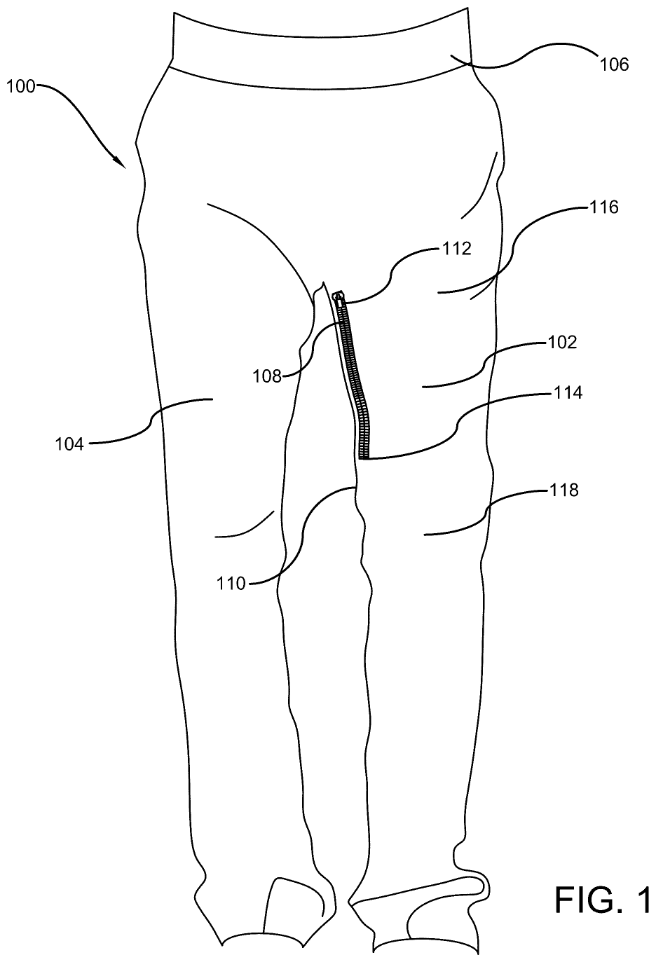

Referring initially to the drawings, FIG. 1 illustrates a perspective view of the catheter-friendly pair of pants of the present invention in accordance with the disclosed structure. The catheter-friendly pair of pants 100 of the present invention is designed to facilitate the use of a catheter for medical purposes and is designed for individuals who require catheterization, such as those with chronic bladder conditions, spinal cord injuries, or post-surgical needs. The improved pair of pants 100 provides maximum comfort by preventing the catheter tube from causing friction against the skin or becoming tangled with clothing. More specifically, the pair of pants 100 include a first leg member 102 and a second leg member 104. The leg members 102, 104 extend vertically downwards from the top end or waist member 106. The leg members 102, 104 are designed to accommodate legs of a wearer of the pants 100. The pair of pants 100 includes a zipper 108 into the inner seam 110 of one of the leg members (102 in the present embodiment).

In the preferred embodiment, the zipper 108 has a length from about 6 inches to about 12 inches, depending on the length of the leg members 102, 104 and zipper 108 is more preferably has a length of from 6 to 12 inches extending between the top end 112 and the bottom end 114. The length of the zipper 108 is chosen to provide sufficient space for a catheter tube to pass therethrough without causing discomfort to the wearer. The top end 112 is disposed near the thigh portion 116 of the leg member 102 and the bottom end 114 is disposed near the knee covering portion or middle portion 118 of the leg member 102.

The zipper 108 is located on the inner seam 110 enabling the zipper 108 to be maintained out of sight, thereby maintaining the appearance of regular pants and keeping the medical condition of the wearer private. The pair of pants 100 can be worn to work or while traveling without affecting use of a catheter. The pair of pants 100 can be any length and size and can be made of any type of antimicrobial and moisture-wicking fabrics such as cotton, polyester, denim, wool, corduroy, leather, blends thereof and the like. The concealed inner seam fastening system catheter-accessible trousers 100 are designed to accommodate different types of catheters such as Foley, intermittent, and suprapubic catheters.

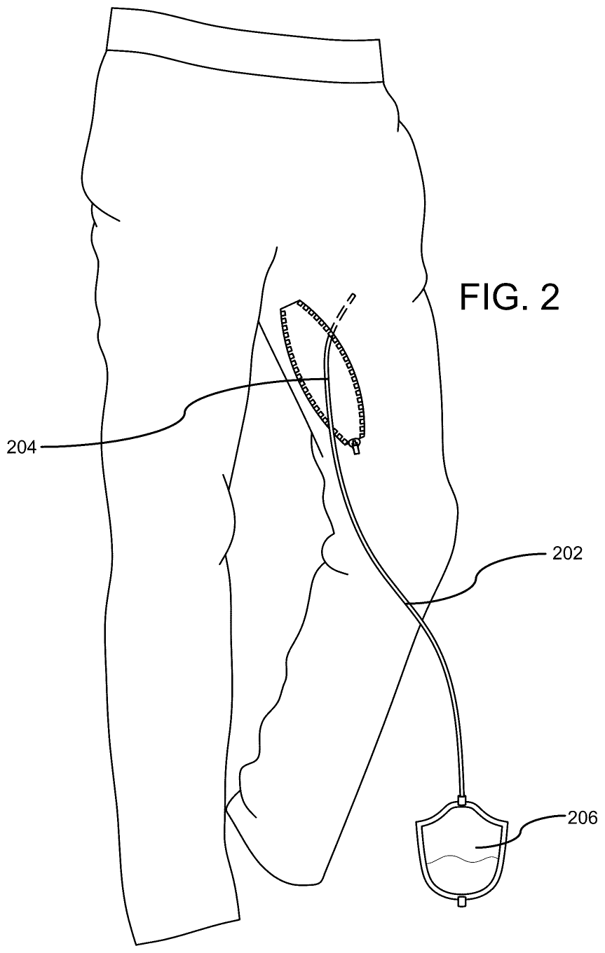

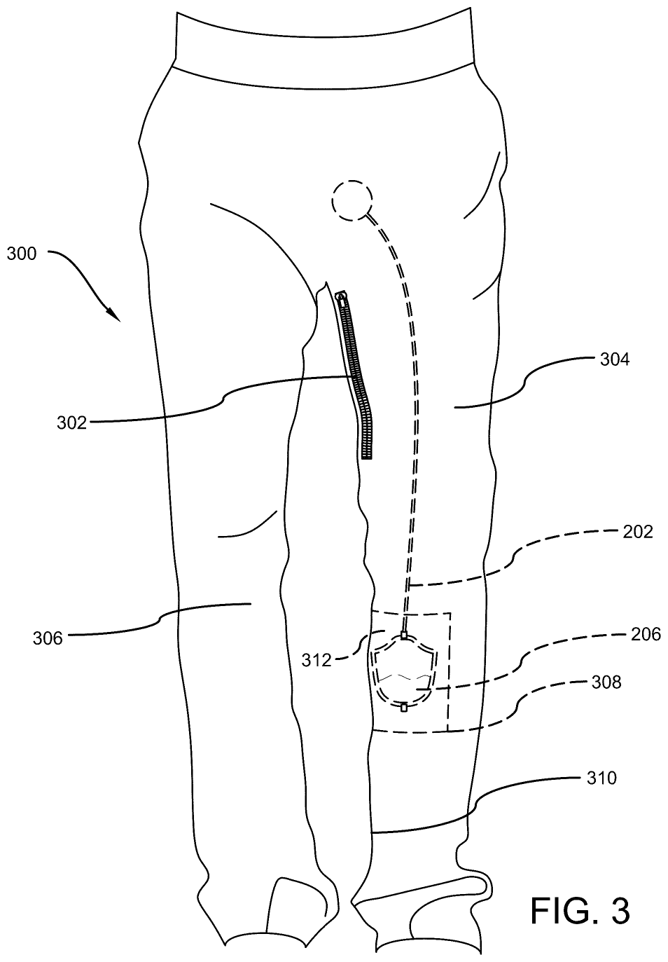

FIG. 2 illustrates a perspective view showing use of the catheter-friendly pair of pants of the present invention in accordance with the disclosed structure. In use, the pair of pants 100 are donned like conventional trousers. Once the pair of pants 100 is donned, a catheter tube 202 is guided through the inner seam zipper opening or access space 204 to exit the leg member 102. For accessing the opening or access space 204, the zipper 108 is unfastened to an open position. A catheter bag 206 is attached to the free end 208 of the catheter tube 202 and the bag 206 can be placed outside the pants and can be attached to the leg of the wearer or can be placed inside. In some embodiments, the pair of pants 100 include an interior pocket for accommodating or holding the catheter bag 206 as illustrated in FIG. 3. The zipper 108 can be partially closed to secure the catheter tube 202 in place while leaving space for the tube 202 to move freely without being pinched.

FIG. 3 illustrates a perspective view showing another embodiment of the catheter-accessible trousers of the present invention in accordance with the disclosed structure. In the present embodiment, the pair of pants 300 include a zipper 302 on at least one leg member 304 or 306 and an internal pocket 308 is disposed on the inner surface 310 of the leg member 304 for accommodating or holding the catheter bag 206. The zipper 302 is opened to place the catheter bag 206 into the pocket 308 or accessing the pocket 308 for removing the bag 206 when the catheter bag needs to be drained. In one embodiment, the member 312 of the leg member which covers the pocket 308 can be transparent, thereby functioning as an inspection window enabling users to check the bag's fill level without needing to remove the bag from the pants. The pocket 308 can be made of natural materials, such as cotton, hemp, and the like or can also be made of synthetic materials, such as, polymers, foam, sponges, or any other synthetic fabric. The pipe 202 can also be passed through a channel (not shown) disposed along the inner surface of the leg member 304, thereby preventing contact with skin of the wearer of the pants.

It will be apparent to a person skilled in the art that in different embodiments of the catheter-friendly pair of pants, the pants are not required to be fully removed to access the catheter tube or remove the catheter bag. The design and position of the inner seam zipper keep the catheter system discreet, enabling the wearer to go about their daily activities with minimal visibility of the catheter tube. The pair of pants 100, 300 enable wearers to engage in daily activities with minimal inconvenience, improving their overall lifestyle.

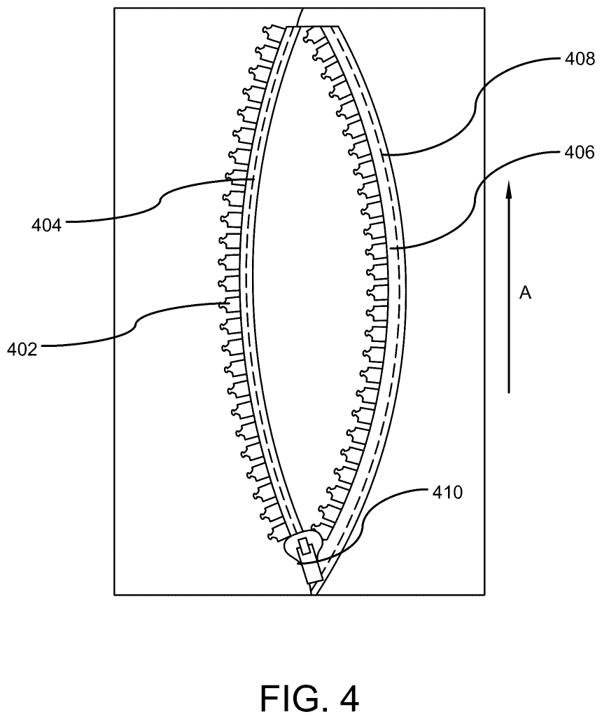

FIG. 4 illustrates an enlarged view of the inner seam zipper used in the catheter-accessible trousers in accordance with the disclosed structure. As illustrated, a zipper male component 402 is sewn along a first edge 404 of the inner seam of the pants 100 and a zipper female component 406 is sewn along a second edge 408 of the inner seam. When the zipper tab 410 is maneuvered in the direction indicated by Arrow A, the male component 402 and the female component 404 are fastened to close the zipper and can be pulled in opposite direction to open the zipper.

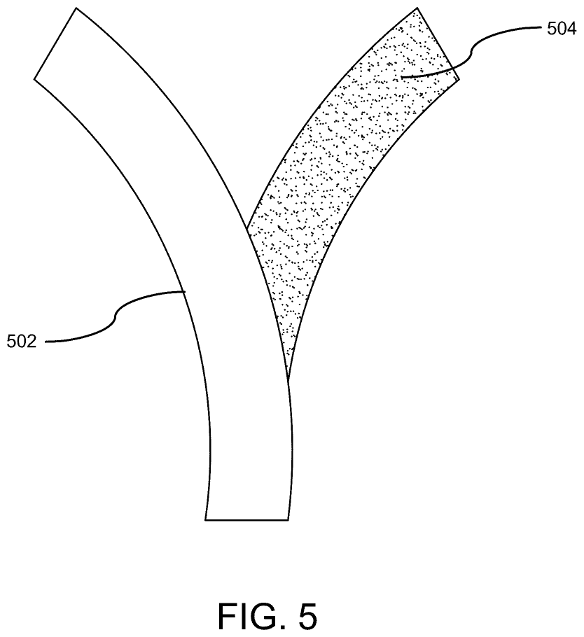

FIG. 5 illustrates a perspective view showing another embodiment of fastener used in the concealed inner seam fastening system catheter-accessible trousers of the present invention in accordance with the disclosed structure. As illustrated, a hook fastener 502 and a corresponding loop fastener 504 can be used in place of the zipper along the inner scam of the pants for creating an opening for guiding the catheter tube. A selected portion of the hook and loop fastener can be fastened to secure the catheter tube 202 in place while leaving space for the tube 202 to move freely. In some embodiments, any other type of fastener such as a snap-button fastener, a pressure adhesive, a magnetic fastener or any other type of fastener can be used along the inner seam of the pants.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “catheter-accessible trousers”, “catheter-friendly pair of pants”, “concealed inner seam fastening system catheter-accessible trousers”, and “pair of pants” are interchangeable and refer to the catheter-accessible trousers with integrated fastener 100 of the present invention.

Notwithstanding the forgoing the catheter-accessible trousers with integrated fastener 100 of the present invention can be of any suitable size and configuration as is known in the art without affecting the overall concept of the invention, provided that it accomplishes the above stated objectives. One of ordinary skill in the art will appreciate that the catheter-accessible trousers with integrated fastener 100 as shown in the FIGS. are for illustrative purposes only, and that many other sizes and shapes of the catheter-accessible trousers with integrated fastener 100 are well within the scope of the present disclosure. Although the dimensions the catheter-accessible trousers with integrated fastener 100 are important design parameters for user convenience, the catheter-accessible trousers with integrated fastener 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An accessible pair of pants comprising:
a pair of pants having a waist member;
a first leg member;
a second leg member;
an access space; and
a zipper;
wherein said first leg member and said second leg member extending vertically downward from said waist member;
wherein said first leg member and said second leg member accommodate a first leg and a second leg of a wearer of said pair of pants;
wherein an inner seam of at least one of said first leg member and said second leg member having said access space and said zipper;
wherein said zipper selectively opens and closes said access space; and
further wherein said access space having a catheter tube passing therethrough.

2. The accessible pair of pants of claim 1, wherein said zipper having a top end and a bottom end aligned with said inner seam.

3. The accessible pair of pants of claim 2, wherein said top end disposed proximal to a thigh portion of said at least one of said first leg member and said second leg member.

4. The accessible pair of pants of claim 3, wherein said bottom end disposed proximal to a knee covering portion of said at least one of said first leg member and said second leg member.

5. The accessible pair of pants of claim 4, wherein said catheter tube is selected from the group consisting of a Foley catheter, an intermittent catheter, and a suprapubic catheter.

6. The accessible pair of pants of claim 5, wherein said catheter tube connected to a catheter bag.

7. The accessible pair of pants of claim 6, wherein said catheter bag is outside of said at least one of said first leg member and said second leg member.

8. The accessible pair of pants of claim 6, wherein said catheter bag is inside of said at least one of said first leg member and said second leg member.

9. A catheter accessible pair of pants comprising:
a pair of pants having a waist member;
a first leg member;
a second leg member;
an access space;
a zipper;
a catheter tube; and
a catheter bag;
wherein said first leg member and said second leg member extending vertically downward from said waist member;
wherein said first leg member and said second leg member accommodate a first leg and a second leg of a wearer of said pair of pants;
wherein an inner seam of at least one of said first leg member and said second leg member having said access space and said zipper;
wherein said zipper selectively opens and closes said access space;
wherein said access space providing an opening for a catheter tube to pass therethrough;
wherein said catheter tube passes through said access space; and
further wherein said catheter tube connected to said catheter bag.

10. The accessible pair of pants of claim 9, wherein said zipper having a top end and a bottom end aligned with said inner seam.

11. The accessible pair of pants of claim 10, wherein said top end disposed proximal to a thigh portion of said at least one of said first leg member and said second leg member.

12. The accessible pair of pants of claim 11, wherein said bottom end disposed proximal to a knee covering portion of said at least one of said first leg member and said second leg member.

13. The accessible pair of pants of claim 12, wherein said catheter tube is selected from the group consisting of a Foley catheter, an intermittent catheter, and a suprapubic catheter.

14. The accessible pair of pants of claim 9, wherein said catheter bag is outside of said at least one of said first leg member and said second leg member.

15. The accessible pair of pants of claim 9, wherein said catheter bag is inside of said at least one of said first leg member and said second leg member.

16. The accessible pair of pants of claim 15, wherein said at least one of said first leg member and said second leg member having an interior pocket for holding said catheter bag.

17. A catheter accessible pair of pants comprising:
a pair of pants having a waist member;
a first leg member;
a second leg member;
an access space;
a fastener;
a catheter tube; and
a catheter bag;
wherein said first leg member and said second leg member extending vertically downward from said waist member;
wherein said first leg member and said second leg member accommodate a first leg and a second leg of a wearer of said pair of pants;
wherein an inner seam of at least one of said first leg member and said second leg member having said access space and said fastener;
wherein said fastener selectively opens and closes said access space;
wherein said access space providing an opening for a catheter tube to pass therethrough;
wherein said catheter tube passes through said access space; and
further wherein said catheter tube connected to said catheter bag.

18. The accessible pair of pants of claim 17, wherein said fastener is selected from the group consisting of a zipper, a hook and loop fastener, a snap-button fastener, a pressure adhesive, and a magnetic fastener.

19. The accessible pair of pants of claim 17, wherein said at least one of said first leg member and said second leg member having an interior pocket for holding said catheter bag.

20. The accessible pair of pants of claim 19, wherein said catheter bag is inside of said at least one of said first leg member and said second leg member.
